(19) United States
(12) Patent Application Publication
(10) Pub. No.: US 2019/0274852 A1
(43) Pub. Date: Sep. 12, 2019

(54) BIO-COMPATIPLE VASCULAR IMPLANT

(71) Applicant: pfm Medical, Inc., Carlsbad, CA (US)

(72) Inventor: Jiaye Jho, Carlsbad, CA (US)

(21) Appl. No.: 16/297,921

(22) Filed: Mar. 11, 2019

Related U.S. Application Data
(60) Provisional application No. 62/641,603, filed on Mar. 12, 2018.

(51) Int. Cl. A61F 2/90; A61L 27/06

(52) U.S. Cl. CPC A61F 2/90; A61L 31/16; A61L 27/06

(57) ABSTRACT

A vascular implant, such as a stent, is configured with a surface of biocompatible material, such as titanium. The material of the stent is polypropylene upgraded with a surface of titaniferous layer that is provided on the stent via a plasma-assisted chemical vapour deposition process.

### REFERENCE TO PRIORITY DOCUMENT

The present application claims priority to co-pending U.S. Provisional Patent Application Ser. No. 62/641,603 entitled “Bio-Compatible Vascular Implant”, filed Mar. 12, 2018. Priority to the filing date is claimed and the provisional application is incorporated herein by reference in its entirety.

### BACKGROUND

A stent is a tubular implant that can be positioned inside an anatomical body lumen of a patient, such as inside a blood vessel, canal, or duct to aid healing, provide structural support, and/or relieve an obstruction. The stent is desirably made of a material that has the structural characteristics to properly support the vessel while also having material properties that are bio-compatible.

Given that the stent is a foreign object, there is a risk that the patient's body may reject the stent after placement. In view of the foregoing, there is a need for stents that reduce the likelihood that a patient's body will reject the stent as non-biocompatible.

### SUMMARY

Disclosed herein is a vascular implant, such as a stent, that is configured with a surface of biocompatible material, such as titanium. In an embodiment, the material of the stent is polypropylene upgraded with a surface of titaniferous layer that is provided on the stent via a plasma-assisted chemical vapour deposition process.

In one aspect, there is disclosed a stent assembly adapted to be implanted in a blood vessel, comprising body formed of a plurality of interconnected struts that are attached to one another to form a plurality of cells or openings, wherein at least a portion of the body comprises polypropylene upgraded with a surface of titaniferous layer, the titaniferous layer being provided on the stent via a plasma-assisted chemical vapour deposition process.

In another aspect, there is disclosed a method of manufacturing a vascular stent assembly, comprising: forming a body of a plurality of interconnected struts that are attached to one another to form a plurality of cells or openings, wherein at least a portion of the body comprises polypropylene; and applying a plasma-assisted chemical vapour deposition process to the body so as to form a surface of titaniferous layer on at least one of the struts

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

The details of one or more variations of the subject matter described herein are set forth in the accompanying drawings and the description below. Other features and advantages of the subject matter described herein will be apparent from the description and drawings, and from the claims.

FIG. 1 shows an example implant comprising a stent.

FIG. 2 shows a schematic representation of a process for forming a metal containing layer on a stent. Reference: Frey, H. Vakuumbeschichtung (1995) 1. Düsseldorf: VDI-Verlag.

FIG. 3 shows a schematic representation of a material layer structure of the stent.

### DETAILED DESCRIPTION

Before the present subject matter is further described, it is to be understood that this subject matter described herein is not limited to particular embodiments described, as such may of course vary. It is also to be understood that the terminology used herein is for the purpose of describing particular embodiments only, and is not intended to be limiting. Unless defined otherwise, all technical terms used herein have the same meaning as commonly understood by one skilled in the art to which this subject matter belongs.

Disclosed herein is a vascular implant, such as a stent, that is configured with a surface of biocompatible material, such as titanium. In an embodiment, the material of the stent is polypropylene upgraded with a surface of titaniferous layer that is provided on the stent via a plasma-assisted chemical vapour deposition process.

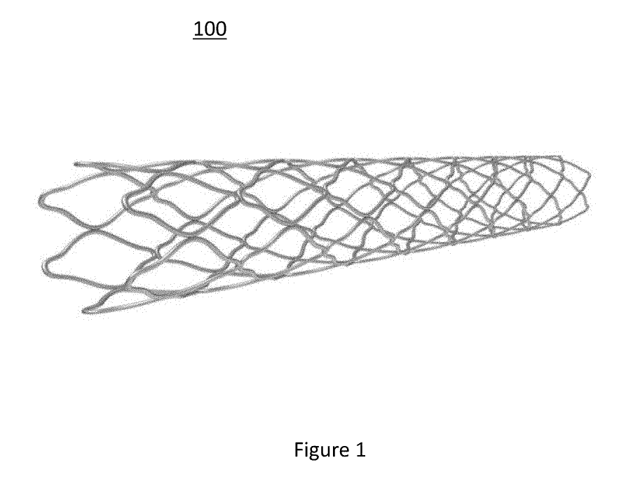

FIG. 1 shows a stent 100 for positioning in a body lumen. In an embodiment, the stent has a cylindrical or substantially cylindrical shape that extends along a longitudinal axis. The stent 100 defines an outer wall and two open openings on opposite ends of the stent. The stent can be formed of any of a variety of structures including a braided material comprising components arranged in both a longitudinal direction and in a transverse direction (thus longitudinal and/or circumferential direction). The structure of the stent can also be any type of woven wire, knitted wire and/or braided wire. The wall of the stent can also be made of wire mesh. It should be appreciated that the structure and material of the stent may vary.

The stent can have a body formed of a plurality of interconnected struts or wires that are attached to one another to form a plurality of cells or openings. The struts can be attached to one another in any of a variety of manners. The stent body generally forms a cylindrical shape that is sized and shaped to fit within a blood vessel.

In an embodiment, the stent 100 is made of a polypropylene that has been upgraded with a titanium containing amorphous layer. In this regard, the biocompatibility of the polypropylene stent is treated to produce a surface of titanium carboxynitride. Pursuant to the process described herein, the physical properties of the polypropylene are retained and the surface treatment improves the biocompatibility. A plasma-assisted chemical vapour deposition (PACVD) process is used to upgrade the polymer of the stent. The PACVD process works with assistance of metal organics that decompose at a predetermined reaction temperature, such as at approximately 30-35° C., which is relatively low. The metal organics release a titanium containing layer onto the surface of the stent.

Due to a relatively low reaction temperature, the properties of the polypropylene are unchanged. That is, the polypropylene retains its tensile strength and flexibility. This process generates a titanium containing layer on the stent in the nanometer range, which is very thin.

The surface layer of titanium carboxynitride increases the biocompatibility of the stent. Due to the extremely thin layer, the flexibility and tensile strength of the polypropylene are retained. The stent implant is therefore a combination of two materials, a flexible polypropylene portion and a biocompatible titanium containing layer.

The formation of the titanium containing layer onto the polypropylene is achieved pursuant to the process described below. The layer is formed in a plasma upgrading technique via a chemical reaction that is divided into three stages:

1) Transport of starting materials to the reaction zone;

2) Conversion; and

3) Removal of any unwanted reaction products.

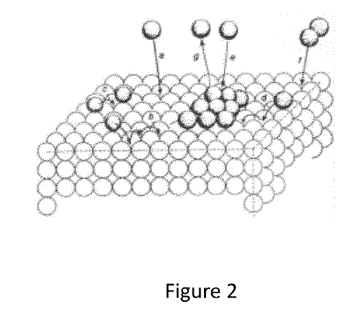

During the reaction process, atoms or molecules impinge upon a polymer substrate wherein the polymer substrate is the stent material. The reaction between the starting materials and the adsorption of the final product achieves a stable state that is characterized by a minimum of free enthalpy. This stable state is not reached immediately as the random site of impact is not the insertion site. The processes involved in the formation of the upgrade are graphically expressed in FIG. 2.

With reference to FIG. 2, in a first step (a) of the process, particles are adsorbed from a gas chamber. In step (b) the particles diffuse on the surface of a substrate such as a polypropylene substrate. In step (c), adsorbed particles cluster to form dimers, which results in growth of clusters of adsorbed particles via surface diffusion in step (d). Next, the clusters grow via adsorption of particles from the gas phase in step (e) and dimers are adsorbed in step (f). Particles are then desorbed in step (g).

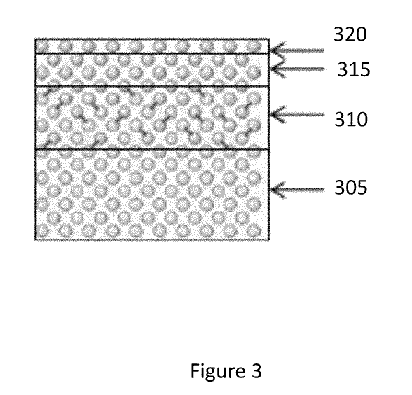

The resultant material of the stent has a structure that is divided into four different zones, as shown in FIG. 3. The structure includes an interior substrate material 305 and a transition zone 310 positioned between the substrate 305 and an outer layer 315, which includes a surface 320 that is in contact with the environment. The transition zone, which forms differently depending on the chemical composition, the formation parameters and the substrate material, determines the bonding strength between the titanium containing layer and polypropylene.

For the production of integrated material bonds by upgrading a synthetic polymer component, the adherent bond is an essential prerequisite. The bond between two bodies is characterized by the force joining the two bodies together. The most important criterion here is adhesion. It characterizes the force acting on the surfaces of the two bodies. Adhesion forces arise from the superimposition of chemical, physical and mechanical interactions at the boundary layers of the joined parts.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. However, such terms are provided to establish relative frames of reference, and are not intended to limit the use or orientation of the systems to a specific configuration described in the various implementations.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. A stent assembly adapted to be implanted in a blood vessel, comprising:
body formed of a plurality of interconnected struts that are attached to one another to form a plurality of cells or openings, wherein at least a portion of the body comprises polypropylene upgraded with a surface of titaniferous layer, the titaniferous layer being provided on the stent via a plasma-assisted chemical vapour deposition process.

2. A stent assembly as in claim 1, wherein at least a portion of the body comprises an outer surface of titanium carboxynitride.

3. A stent assembly as in claim 1, wherein at least a portion of the body comprises both a flexible polypropylene portion and a biocompatible titanium layer.

4. A stent assembly as in claim 1, wherein the body forms a cylindrical shape.

5. A stent assembly as in claim 1, wherein the struts comprise a braided material.

6. A stent assembly as in claim 1, wherein the struts extend longitudinally and transversely relative to along axis of the stent.

7. A stent assembly as in claim 1, wherein the plasma-assisted chemical vapour deposition process works with assistance of metal organics that decompose at a predetermined reaction temperature.

8. A stent assembly as in claim 7, wherein the metal organics release a titanium containing layer onto the surface of the stent assembly.

9. A method of manufacturing a vascular stent assembly, comprising:
forming a body of a plurality of interconnected struts that are attached to one another to form a plurality of cells or openings, wherein at least a portion of the body comprises polypropylene; and
applying a plasma-assisted chemical vapour deposition process to the body so as to form a surface of titaniferous layer on at least one of the struts.

10. A method as in claim 9, wherein the plasma-assisted chemical vapour deposition process works with assistance of metal organics that decompose at a predetermined reaction temperature.

11. A method as in claim 10, wherein the metal organics release a titanium containing layer onto the surface of the stent assembly.

12. A method as in claim 9, wherein at least a portion of the body comprises an outer surface of titanium carboxynitride.

13. A method as in claim 9, wherein at least a portion of the body comprises both a flexible polypropylene portion and a biocompatible titanium layer.

14. A method as in claim 9, wherein the body forms a cylindrical shape.

15. A method as in claim 9, wherein the struts comprise a braided material.
